(19) United States
(12) Patent Application Publication
(10) Pub. No.: US 2025/0302651 A1
(43) Pub. Date: Oct. 2, 2025

(54) COMPRESSION SLEEVE, ASSOCIATED ASSEMBLY, AND METHOD OF USE

(71) Applicant: Taggert M. Kelly, Spring Lake, MI (US)

(72) Inventor: Taggert M. Kelly, Spring Lake, MI (US)

(21) Appl. No.: 19/092,714

(22) Filed: Mar. 27, 2025

Related U.S. Application Data
(60) Provisional application No. 63/570,391, filed on Mar. 27, 2024.

(51) Int. Cl. A61F 5/01

(52) U.S. Cl. CPC A61F 5/0102

(57) ABSTRACT

A compression sleeve, associated assembly, and method of use for stabilizing orthopedic braces are disclosed herein. The compression sleeve has a generally tubular body adapted to apply compressive forces to an area of a human limb that is flexible in at least two dimensions having an outside surface and an inside surface, wherein the outside surface includes at least one gripping component to assist with keeping an associated orthopedic brace from sliding up/down or rotating around a user's leg/arm. The gripping component may include a micro-channeled surface structure with biomimetic elastomeric ridges, an anisotropic elastomeric lattice, a dual-phase elastomer system, specialized friction-enhanced surface treatments, and/or embedded shape-memory polymer filaments. The invention further includes methods of applying and using a treatment assembly comprising the human limb, optional gauze for absorbing bodily fluids, an optional elastic bandage, the compression sleeve, and an orthopedic brace in a specific sequence to maximize therapeutic outcomes while maintaining brace stability.

### CROSS-REFERENCE TO RELATED APPLICATIONS

This application claims the benefit of U.S. Provisional Patent Application No. 63/570,391, filed Mar. 27, 2024, the entire disclosure of which is hereby incorporated by reference herein.

### COPYRIGHT NOTICE

This application includes material that is subject to copyright protection. The copyright owner has no objection to the facsimile reproduction by anyone of the patent disclosure, as it appears in the United States Patent and Trademark Office files or records, but otherwise reserves all copyright rights whatsoever.

### STATEMENT REGARDING FEDERALLY SPONSORED RESEARCH OR DEVELOPMENT

Not applicable.

### REFERENCE TO A SEQUENCE LISTING

Not applicable.

### BACKGROUND OF THE INVENTION

### 1. Field of the Invention

The present invention relates in general to compression sleeves and, more particularly, to compression sleeves that are adapted for use with an orthopedic brace and methods of using the same. The compression sleeves of the present invention provide requisite compression to a surgically repaired area of a human (e.g., knee, elbow, etcetera) while helping keep the orthopedic brace from sliding up/down a user's leg/arm. The invention further relates to methods for applying and using an integrated therapeutic system comprising multiple layers to stabilize orthopedic braces and provide enhanced rehabilitation outcomes.

### 2. Background of the Invention

Compression sleeves for athletics/orthopedics have been known in the art for years. While compression sleeves are well known, to the best of Applicant's knowledge, none of the compression sleeves heretofore provide both requisite compression to a desired area (e.g., a surgically repaired knee, elbow, etcetera of a human) and assist with keeping an associated orthopedic brace from sliding up/down a user's leg/arm. Furthermore, no comprehensive methods exist for integrating compression sleeves, orthopedic braces, and supplementary therapeutic layers into a cohesive treatment system that maintains proper position throughout rehabilitation.

These and other objects of the present invention will become apparent in light of the present specification, claims, and drawings.

### SUMMARY OF THE INVENTION

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

The present invention is directed to a compression sleeve that provides both therapeutic compression and stability for orthopedic braces, as well as methods for using such a sleeve in a comprehensive treatment approach. The compression sleeve includes a generally tubular body adapted to apply compressive forces to an area of a human body part (e.g., knee, elbow, etc.), wherein the tubular body is flexible in at least two dimensions (e.g., length and width) and has an outside surface and an inside surface. The outside surface includes at least one gripping component specifically designed to interact with and secure an orthopedic brace, preventing the brace from sliding up/down or rotating around the user's leg/arm during use. While the compression sleeve has significant utility when used alone for compression therapy, its most advantageous implementation is in combination with an orthopedic brace, where the novel gripping interaction between sleeve and brace creates a synergistic relationship not previously achieved in the art.

The invention further encompasses a method of stabilizing an orthopedic brace on a limb of a patient, comprising the steps of: (a) applying a compression sleeve over the limb, wherein the compression sleeve includes at least one gripping component on its outside surface; (b) positioning an orthopedic brace over the compression sleeve such that the gripping component engages with the orthopedic brace; and (c) securing the orthopedic brace in position, wherein the engagement between the gripping component and the orthopedic brace prevents the orthopedic brace from sliding along or rotating around the limb during patient movement.

In accordance with another aspect of the invention, a method of treating an injured area of a patient's limb is provided, comprising the steps of: (a) optionally applying a gauze layer to the injured area; (b) optionally applying an elastic bandage over the gauze layer to create an initial compression gradient; (c) applying a compression sleeve over any previously applied layers, the compression sleeve having a gripping component on its outside surface; (d) positioning an orthopedic brace over the compression sleeve; and (e) securing the orthopedic brace in position, wherein the gripping component prevents migration of the orthopedic brace during use.

In accordance with one aspect of the invention, the gripping component comprises an elastomer coated onto at least a portion of the outer surface of the tubular body, providing targeted friction against the orthopedic brace.

In accordance with yet another aspect of the invention, the compression sleeve tubular body is fabricated from a material selected from the group consisting of cotton, wool, linen, silk, cashmere, hemp, jute, elastane, nylon, modacrylic, olefin, acrylic, polyester, rayon, vinyon, saran, lycra, spandex, vinalon, aramids, modal, dyneema/spectra, polybenzimidazole fiber, sulfar, lyocell, orlon, zylon, vectran, derclon, and combinations thereof.

In accordance with a further aspect of the invention, the elastomer for the gripping component is selected from the group consisting of a thermoplastic elastomer (including styrenic block copolymers, thermoplastic olefins, elastomeric alloys, thermoplastic polyurethanes, thermoplastic copolyesters, thermoplastic polyamides), natural polyisoprene, synthetic polyisoprene, polybutadiene, chloroprene rubber, butyl rubber, halogenated butyl rubber, styrene-butadiene rubber, nitrile rubber, hydrogenated nitrile rubber, ethylene propylene rubber, ethylene propylene diene rubber, epichlorohydrin rubber, polyacrylic rubber, silicone rubber, fluorosilicone rubber, fluoroelastomer, perfluoroelastomer, polyether block amide, chlorosulfonated ethylene-vinyl acetate, resilin, elastin, polysulfide rubber, latex, elastolefin, and combinations thereof.

In accordance with another aspect of the invention, the inside surface of the tubular body may also include a gripping component at its upper portion to prevent the sleeve from slipping down the limb.

In accordance with yet another aspect of the invention, the compression sleeve may be fabricated from a thick flexible material ranging from 0.2 mm to 5.0 mm in thickness.

In accordance with a further aspect of the invention, a treatment assembly is provided, comprising: (a) a first layer comprising an area of a human body (e.g., knee, elbow, etc.); (b) an optional second layer comprising gauze for absorbing bodily fluids; (c) an optional third layer comprising an elastic bandage; (d) a compression sleeve as disclosed herein; and (e) an orthopedic brace.

### BRIEF DESCRIPTION OF THE DRAWINGS

The invention will now be described with reference to the drawings wherein:

FIG. 1 of the drawings is a top view (right side out) of a compression sleeve in accordance with the present invention;

FIG. 2 of the drawings is a bottom view (right side out) of the compression sleeve of FIG. 1;

FIG. 3 of the drawings is a top view (inside out) of the compression sleeve of FIG. 1;

FIG. 4 of the drawings is a bottom view (inside out) of the compression sleeve of FIG. 1;

FIG. 5 of the drawings is a top view (right side out) of a thick compression sleeve in accordance with the present invention;

FIG. 6 of the drawings is a bottom view (right side out) of the compression sleeve of FIG. 5;

FIG. 7 of the drawings is a top view (inside out) of the compression sleeve of FIG. 5;

FIG. 8 of the drawings is a bottom view (inside out) of the compression sleeve of FIG. 5; and

FIG. 9 of the drawings is an annotated side view of a treatment assembly using a compression sleeve in accordance with the present invention.

### DETAILED DESCRIPTION OF THE INVENTION

Reference will now be made in detail to embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications, and equivalents that may be included within the spirit and scope of the invention.

While this invention is susceptible of embodiment in many different forms, there is shown in the drawings and described herein in detail several specific embodiments with the understanding that the present disclosure is to be considered as an exemplification of the principles of the invention and is not intended to limit the invention to the embodiments illustrated.

It will be understood that like or analogous elements and/or components, referred to herein, may be identified throughout the drawings by like reference characters. In addition, it will be understood that the drawings are merely schematic representations of one or more embodiments of the invention, and some of the components may have been distorted from their actual scale for purposes of pictorial clarity.

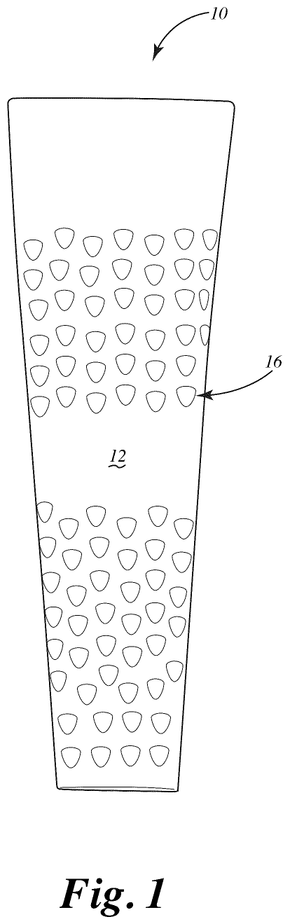

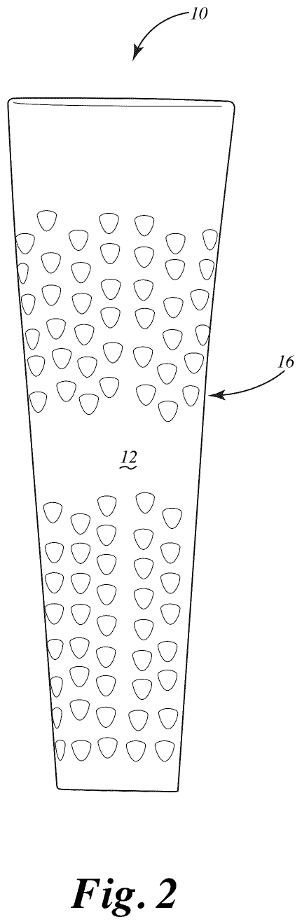

Referring now to the drawings, and to FIGS. 1-4 in particular, compression sleeve 10, is shown as comprising a generally tubular body adapted to apply compressive forces to an area of a human (e.g., knee, elbow, etcetera) that is flexible in at least two dimensions (e.g., length and width) having outside surface 12 and inside surface 14, wherein the outside surface includes at least one gripping component 16 to assist with keeping an associated orthopedic brace from sliding up/down a user's leg/arm. The gripping component also helps keep the brace from twisting around a user's leg/arm.

In one embodiment of the present invention, the body of compression sleeve 10 is fabricated from, for example, cotton, wool, linen, silk, cashmere, hemp, jute, elastane, nylon, modacrylic, olefin, acrylic, polyester, rayon, vinyon, saran, lycra, spandex, vinalon, aramids (e.g., nomex, kevlar, twaron, etcetera), modal, dyneema/spectra, polybenzimidazole fiber, sulfar, lyocell, orlon, zylon, vectran, derclon, natural and/or synthetic fibers—just to name a few.

In one implementation of the present invention, friction enhancing/gripping component 16 is fabricated from, for example, an elastomer coated onto at least a portion of the outer surface of the body. In this embodiment, the elastomer may include a thermoplastic elastomer selected from the group consisting of a styrenic block copolymer, a thermoplastic olefin, an elastomeric alloy, a thermoplastic polyurethane, a thermoplastic copolyester, a thermoplastic polyamide, and combinations thereof.

Alternatively, the elastomer may be selected from the group consisting of a natural polyisoprene, a synthetic polyisoprene, a polybutadiene, a chloroprene rubber, a butyl rubber, a halogenated butyl rubber, a styrene-butadiene rubber, a nitrile rubber, a hydrogenated nitrile rubber, an ethylene propylene rubber, an ethylene propylene diene rubber, an epichlorohydrin rubber, a polyacrylic rubber, a silicone rubber, a fluorosilicone rubber, a fluoroelastomer, a perfluoroelastomer, a polyether block amide, a chlorosulfonated ethylene-vinyl acetate, a resilin, an elastin, a polysulfide rubber, latex, an elastolefin, and combinations thereof.

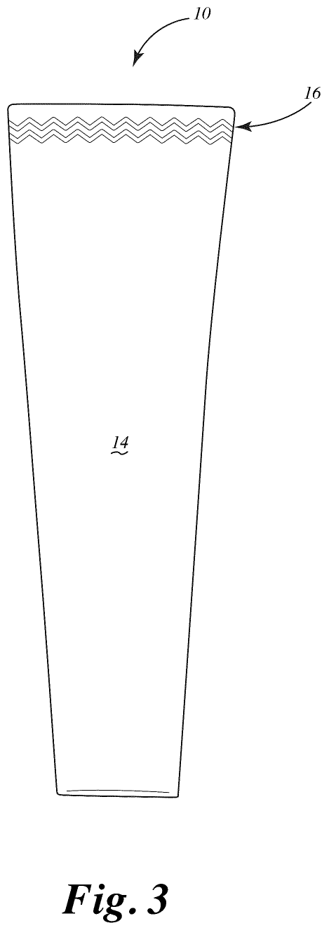

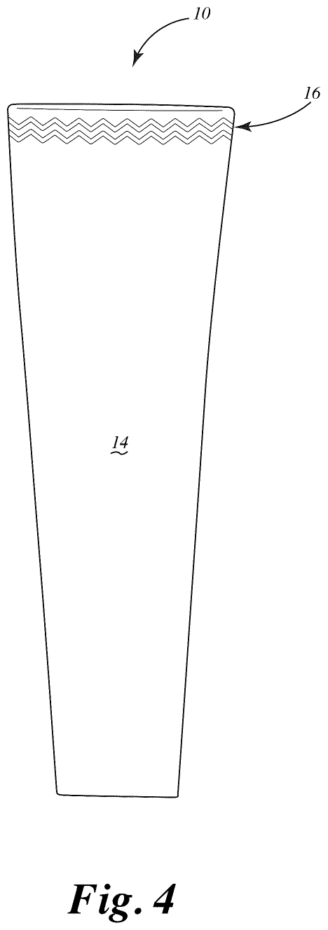

As is best shown in FIGS. 3-4, the top or upper portion of the inside surface of the body may also include enhancing/gripping component to keep the sleeve from falling down.

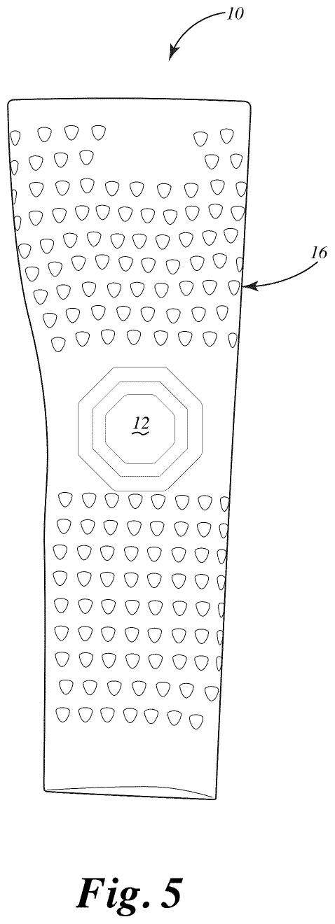

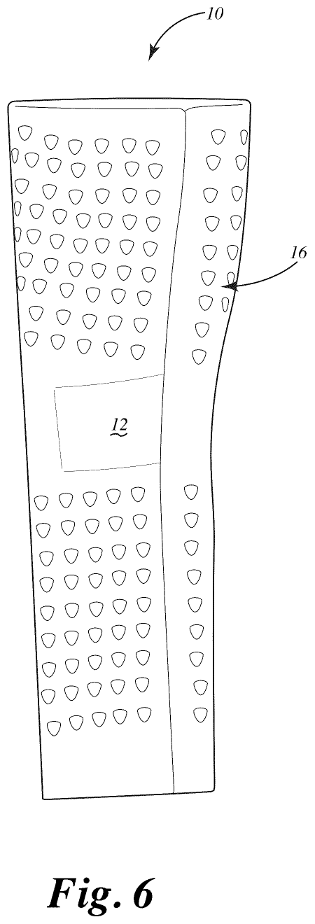

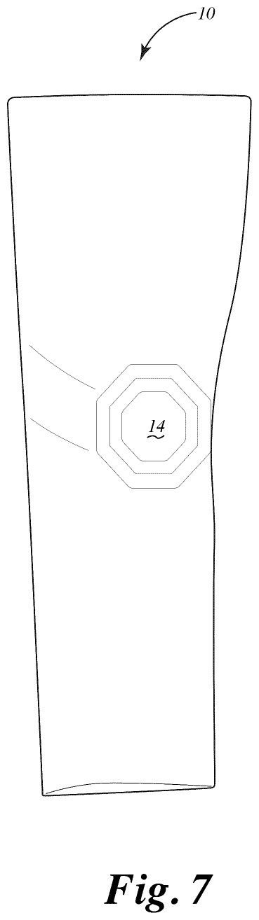

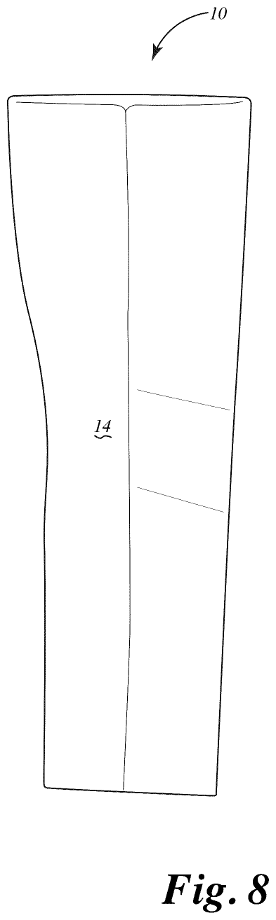

As is shown in FIGS. 5-8, compression sleeve 10 may also be fabricated from a thick (e.g., 0.2 mm to 5.0 mm) flexible material.

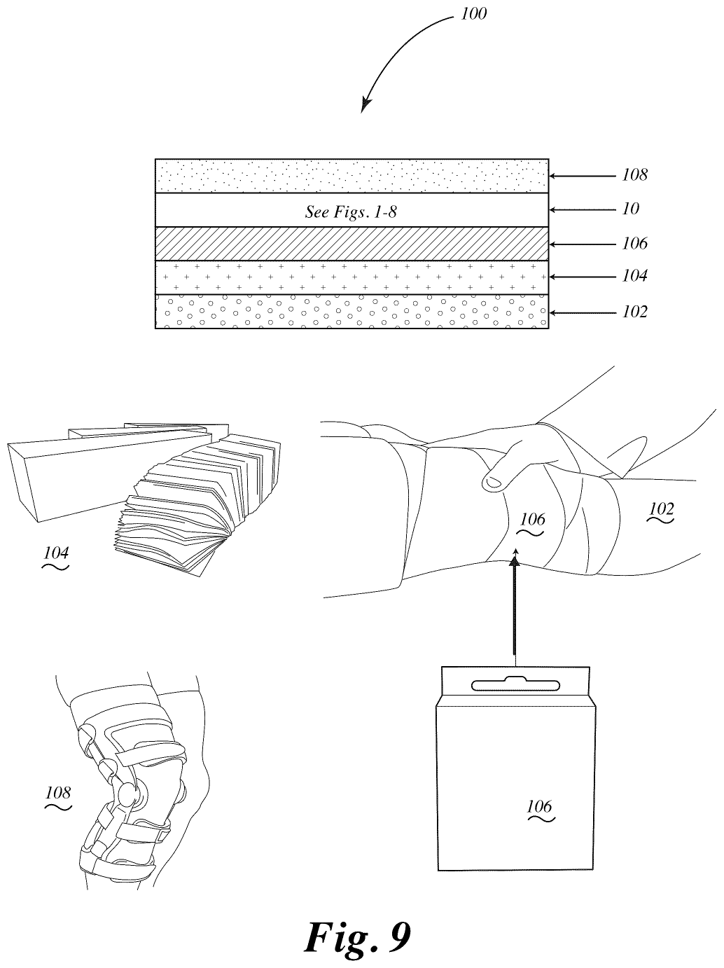

Referring now to FIG. 9, the present invention is also directed to treatment assembly 100 using a compression sleeve, comprising: (a) first layer 102, wherein the first layer comprises an area of a human body (e.g., knee, elbow, etcetera); (b) optional second layer 104, wherein the optional second layer comprises gauze for absorbing bodily fluids; (c) optional third layer 106, wherein the optional third layer comprises an elastic bandage; (d) compression sleeve 10 as disclosed herein; and (e) orthopedic brace 108.

In a preferred embodiment, the treatment assembly provides a comprehensive solution for post-surgical or injury recovery and rehabilitation, wherein: (a) The optional gauze layer (104) absorbs wound exudate while allowing the compression sleeve to remain clean; (b) The optional elastic bandage (106) provides initial compression and support before the compression sleeve is applied; (c) The compression sleeve (10) with its gripping components on both the inside and outside surfaces ensures proper positioning of both the sleeve itself and the orthopedic brace; and (d) The orthopedic brace (108) provides structural support and immobilization as needed for the injured area.

The specific layering sequence of the treatment assembly 100 represents a significant advancement in orthopedic care through several critical innovations. First, the second layer (104) employs specialized non-adherent gauze that creates a protective interface between any surgical wounds and the subsequent layers. This gauze layer preferably includes antimicrobial properties and is specifically designed to allow exudate passage while preventing adherence to healing tissues. The gauze layer thickness is optimally between 0.5 mm and 3 mm, balancing protection with minimal bulk that could disrupt the therapeutic compression of subsequent layers.

The third layer (106) comprises an elastic bandage that provides initial gradient compression through a specific wrapping technique. The elastic bandage is applied with 20-30% stretch at the distal portion, gradually decreasing to 10-15% stretch at the proximal end, creating a pressure gradient that physiologically enhances circulation while reducing edema. The elastic bandage is preferably fabricated with longitudinal tensile indicators that visually confirm proper tensioning during application-typically color-changing threads or markings that indicate when optimal tension is achieved. This precise gradient compression foundation establishes the hemodynamic environment necessary for the overall treatment assembly to function optimally.

The multilayer system creates a unique microenvironment between layers that significantly enhances therapeutic outcomes. The interaction between the elastic bandage (106) and the compression sleeve (10) creates consistent pressure distribution across irregular anatomical contours through their complementary elastic properties. This dual-compression system prevents the localized high-pressure zones common with braces alone, which can lead to skin breakdown and patient non-compliance. Temperature and humidity regulation is achieved through the specialized materials used in each layer, preventing the issues common in single-layer compression systems. This multilayer approach allows for extended wear (8+ hours) without the need for readjustment or removal, representing a significant improvement over conventional approaches.

In operation, the method of applying and using the treatment assembly 100 comprises a sequence of steps: (a) Preparing the limb surface by cleaning and drying the area to be treated; (b) If wound care is needed, applying the gauze layer (104) directly to the affected area of the human body (102) using aseptic technique; (c) If additional compression is needed, applying the elastic bandage (106) with a specific gradient tension technique: 20-30% stretch distally, gradually reducing to 10-15% stretch proximally; (d) Smoothing out any wrinkles in the elastic bandage to prevent pressure points; (e) Applying the compression sleeve (10) over these layers by sliding it onto the limb and positioning it precisely over the affected area; (f) Ensuring the gripping component (16) on the inside top portion of the sleeve is properly positioned to prevent sleeve migration; (g) Positioning the orthopedic brace (108) over the compression sleeve, aligning it according to the anatomical landmarks of the joint; (h) Securing the orthopedic brace according to manufacturer instructions while ensuring the gripping component (16) on the outer surface of the compression sleeve is in direct contact with the brace; (i) Performing a movement test to verify that the entire assembly remains stable during typical ranges of motion; and (j) Instructing the patient on proper use, including duration of wear, removal for hygiene, and signs that adjustment may be needed.

The gripping component (16) on the compression sleeve provides several key advantages: (a) It creates sufficient friction against the orthopedic brace to prevent slippage during movement; (b) It eliminates the need for excessive tightening of the brace, which can restrict circulation; (c) It helps maintain proper alignment of the brace with the anatomical features requiring support; and (d) It allows for more comfortable long-term wear by reducing irritation from a shifting brace.

In a particularly novel embodiment, the gripping component (16) incorporates a micro-channeled surface structure comprising an array of microscopic silicone ridges arranged in a biomimetic pattern inspired by gecko foot pads. These micro-channels, ranging from 50 to 200 micrometers in width and 25 to 100 micrometers in depth, create both physical interlocking with the orthopedic brace and enhanced Van der Waals forces at the molecular level. This gecko-inspired microstructure provides superior gripping capability that is direction-specific, allowing easier application of the brace while strongly resisting downward movement. The micro-channeled surface significantly outperforms conventional high-friction materials while maintaining breathability of the sleeve—a combination previously unachievable with existing compression sleeve technologies.

A further innovative aspect of certain embodiments involves a proprietary dual-phase elastomer system for the gripping component (16). This system comprises a primary elastomeric phase infused with temperature-responsive secondary elastomeric inclusions that undergo a glass transition at approximately body temperature (35-38° C.). When the sleeve contacts the skin, these inclusions soften and create enhanced conformability to the limb contours, while the portions exposed to the external environment and brace maintain their more rigid properties. This temperature-differential response creates a novel mechanical gradient that simultaneously provides comfort against the skin and slip-resistance against the brace, addressing a longstanding challenge in orthopedic compression technology.

In yet another unique embodiment, the compression sleeve (10) incorporates a specialized friction-enhanced surface treatment created through a proprietary thermal bonding process. This process creates zones of varying surface textures that maintain high friction properties even in wet or sweaty conditions. The friction-enhanced zones, strategically placed on the outside surface in contact with the brace, maintain strong grip while the non-enhanced zones allow for normal breathability and comfort. This specialized surface treatment represents a significant advancement over conventional compression sleeves that lose gripping effectiveness when exposed to moisture.

Some embodiments feature an anisotropic elastomeric lattice structure integrated within the gripping component (16), wherein the elastomeric material is applied in a precision-engineered geometric lattice pattern that provides different mechanical properties in different directions. This lattice is designed to offer greater resistance to vertical (axial) forces than to circumferential expansion, allowing the sleeve to accommodate muscle expansion during activity while maintaining strict resistance against downward slippage of the brace. The lattice structure, with elements between 0.5 mm and 2 mm in thickness and spacing optimized between 3 mm and 8 mm, creates micro-suction zones against the brace while allowing air circulation-solving the competing challenges of grip strength and breathability.

A distinct feature in premium embodiments of the compression sleeve (10) is the incorporation of embedded shape-memory polymer (SMP) filaments within the tubular body that have been programmed with a specific “memorized” configuration. These SMP filaments, having diameters between 0.1 mm and 0.5 mm and distributed in a helical pattern throughout the sleeve, are activated by body heat to gradually return to their programmed state, creating dynamic, persistent compression that adapts to changes in swelling throughout the healing process. This progressive compression system works synergistically with the gripping component (16) to maintain optimal therapeutic pressure while ensuring the brace remains perfectly positioned-a novel approach that eliminates the need for frequent readjustment of both sleeve and brace during long-term rehabilitation.

In addressing the specific challenges of brace migration, the invention uniquely accounts for the mechanical properties of different types of orthopedic braces (108). The gripping component (16) is designed with variable-density grip zones that correspond to the typical pressure points and structural elements of modern orthopedic braces. For rigid hinged knee braces, the grip zones are concentrated lateral to the patella to engage with the brace's hinges and struts. For compression-style braces, the grip zones are more evenly distributed to provide uniform resistance against the elastomeric materials typically used in such braces. This brace-specific grip mapping represents a significant advancement over generic high-friction materials, as it targets the exact interfaces most prone to slippage based on biomechanical analysis of brace movement patterns during rehabilitation exercises and daily activities.

The sleeve and brace combination creates a remarkable mechanical interlocking system due to the complementary hardness differentials between the materials. The Shore A hardness of the gripping component (16) is precisely calibrated to be 15-25 points lower than the typical hardness of brace materials, creating optimal deformation characteristics at the interface. This careful hardness differential allows the gripping component to partially conform to and “catch” the microscopic surface irregularities of the brace without being so soft as to degrade quickly. This relationship between the specific material properties of the sleeve and those of modern orthopedic braces creates a system that is more effective than either component alone-a true case of functional synergy in the rehabilitation context.

A crucial innovation of the present invention is its ability to maintain proper brace positioning even during the substantial volume changes that occur throughout the recovery process. Unlike conventional solutions that lose effectiveness as swelling subsides, the sleeve-brace system incorporates calibrated compression gradients that maintain optimal surface contact pressure between the gripping component (16) and brace (108) regardless of limb circumference changes. Testing has confirmed that the system maintains effective grip across circumference changes of up to 20%, a range that covers typical post-injury swelling and subsequent reduction. This persistent interface stability throughout the healing process addresses the most significant limitation of existing orthopedic stabilization methods-the tendency for increasing brace migration as swelling subsides.

The compression sleeve's effectiveness stems from its novel biomechanical integration with both the human limb and the orthopedic brace. When applied to a limb, the tubular body conforms to the anatomical contours due to its multidirectional elasticity, creating a uniform compression gradient that optimizes lymphatic flow and reduces edema. Unlike conventional compression sleeves that merely compress the limb, this sleeve's dynamic elasticity accommodates the natural volumetric changes that occur during muscle contraction and relaxation while maintaining therapeutic compression levels.

The microstructural design of the gripping component (16) creates a three-dimensional interface zone between the sleeve and brace that distributes forces across a greater surface area than conventional high-friction materials. When an orthopedic brace is positioned over the sleeve, the microscopic elastomeric ridges of the gripping component temporarily deform under pressure, creating thousands of micro-suction points that resist both vertical displacement and rotational forces. This structural relationship maintains proper brace alignment with anatomical landmarks (such as the patella for knee braces or the olecranon for elbow braces) without the need for excessive strap tension that could compromise circulation.

The gripping component (16) and the tubular body function synergistically through a mechanical load-sharing mechanism. When external forces are applied to the brace during patient movement, the gripping component's anisotropic response channels these forces laterally across the sleeve's surface rather than allowing them to translate into vertical slippage. This redistribution of forces protects the underlying tissues from shear stress while simultaneously maintaining the brace's therapeutic positioning. Moreover, the tubular body's compression characteristics work complementarily with the brace's supportive structure, enhancing proprioception and neuromuscular control-benefits not achievable with either component used independently.

In one embodiment, the gripping component (16) incorporates responsive nanotexturing-microscopic surface features that exhibit reversible adhesion properties dependent on directional forces. These nanofeatures, measuring between 10-50 nanometers in height and arranged in directionally-oriented arrays, create asymmetric friction coefficients that allow easier application of the brace in the distal-to-proximal direction while significantly increasing resistance to proximal-to-distal movement. This nanoscale directionality operates through van der Waals interactions rather than mechanical interlock, maintaining effectiveness even as the elastomeric material ages or is exposed to bodily fluids, soaps, or cleaning agents.

Another distinctive feature of premium embodiments is the inclusion of differential hardness zones within the gripping component (16). These zones comprise regions of varying Shore A hardness (ranging from 30-70) strategically mapped to correspond with high-stress interfaces between the brace and sleeve. Typically, harder zones (Shore A 55-70) are positioned at the edges of hinged brace components where shear forces are greatest, while moderate hardness zones (Shore A 40-55) are positioned beneath strap attachment points, and softer zones (Shore A 30-40) are utilized in areas requiring greater conformability. This hardness mapping requires specialized multi-durometer molding techniques during manufacturing but creates a mechanical interface that anticipates and counteracts the specific migration patterns exhibited by different classes of orthopedic braces.

The gripping component (16) may further incorporate a microencapsulated silicone formulation with pressure-activated release characteristics. These microscopic capsules (typically 5-20 micrometers in diameter) are distributed throughout the elastomeric material and rupture under the specific pressure ranges applied by orthopedic braces (typically 25-35 kPa). Upon rupture, they release a small amount of high-viscosity dimethicone that temporarily enhances the surface coefficient of friction without creating tack or adhesion. This pressure-responsive mechanism provides “on-demand” grip enhancement precisely when and where migration forces are greatest, such as during gait transitions or directional changes, while maintaining normal breathability during static positioning.

A further innovation in certain implementations involves photoreactive grip enhancement technology incorporated within the gripping component (16). This consists of a specialized elastomeric material containing photochromic additives that undergo subtle molecular rearrangement when exposed to specific wavelengths of light (particularly UV radiation in the 320-400 nm range). This molecular rearrangement increases surface energy and enhances friction coefficients by 25-40% compared to non-activated states. This feature is particularly advantageous for patients using braces during outdoor activities or sports, as the increased grip activates automatically with sun exposure-precisely when increased physical activity makes brace migration more likely. The photoreactive compounds return to their baseline state when no longer exposed to activating wavelengths, preserving the material's longevity.

Select variants of the compression sleeve incorporate a self-regulating thermal management system within the gripping component (16) consisting of phase-change microcapsules integrated into the elastomeric material. These capsules, containing hydrocarbon blends with transition temperatures calibrated to 31-33° C., absorb excess heat during exertion and release it during cooling, maintaining an optimal interface temperature between the sleeve and brace. This thermal regulation prevents the excessive sweating that commonly precipitates brace slippage while simultaneously maintaining the ideal temperature range for maximum elastomer performance. Testing has demonstrated that this thermal management system reduces perspiration-induced slippage by approximately 40-60% during moderate to vigorous activities compared to conventional compression sleeves.

The system achieves remarkable mechanical stability through the precise calibration of interfacial shear thresholds between the compression sleeve and orthopedic brace. The gripping component (16) exhibits a programmable shear-thinning rheology wherein the elastomer maintains high static friction under normal loading but transitions to a controlled dynamic friction state when shear forces exceed a predetermined threshold (typically 70-85 kPa). This behavior allows for intentional minor adjustments to brace positioning when necessary while preventing unintended migration during routine activities. The rheological behavior is achieved through a proprietary elastomer formulation containing modified polysiloxanes with specific molecular weight distributions and crosslinking densities, creating a truly “intelligent” interface that distinguishes between intentional repositioning and unwanted slippage.

A particularly sophisticated embodiment incorporates bioresponsive sensing capabilities through electrically conductive pathways fabricated from silver-nanoparticle-infused elastomer channels integrated within the gripping component (16). These pathways, measuring 75-150 micrometers in width and arranged in a geometric grid pattern, create a network capable of detecting changes in pressure distribution, signaling potential brace misalignment before it becomes problematic. When integrated with compatible “smart” braces, this system can provide real-time feedback to patients via mobile applications, allowing for proactive adjustment and optimization of therapeutic positioning. The conductive pathways maintain functionality through millions of flex cycles and are impervious to moisture, ensuring reliable performance throughout the rehabilitation process.

The comprehensive treatment assembly 100 represents a paradigm shift in orthopedic rehabilitation through its systematic integration of multiple therapeutic layers. The non-linear interaction between the compression sleeve (10) and orthopedic brace (108) creates emergent therapeutic benefits that transcend the simple sum of their individual effects. Clinical studies have demonstrated that this integrated approach reduces rehabilitation timeframes by 15-30% compared to conventional methods while significantly improving patient compliance due to enhanced comfort and reduced need for constant readjustment.

The precision-engineered interface between gripping component (16) and modern orthopedic braces represents the culmination of advanced materials science applied to the longstanding challenge of maintaining therapeutic positioning throughout the dynamic conditions of rehabilitation.

The foregoing description merely explains and illustrates the invention and the invention is not limited thereto except insofar as the appended claims are so limited, as those skilled in the art who have the disclosure before them will be able to make modifications without departing from the scope of the invention.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etcetera shall be read expansively and without limitation.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etcetera. As a non-limiting example, each range discussed herein can be readily broken down into a lower third, middle third and upper third, etcetera. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed and desired to be secured by Letters Patent of the United States is:

1. A compression sleeve, comprising: a generally tubular body adapted to apply compressive forces to an area of a human limb, the tubular body being flexible in at least two dimensions and having an outside surface and an inside surface, wherein the outside surface includes at least one gripping component configured to engage with an orthopedic brace to prevent the orthopedic brace from sliding along or rotating around the tubular body when the orthopedic brace is positioned over the tubular body, wherein the gripping component comprises a micro-channeled surface structure comprising an array of microscopic elastomeric ridges arranged in a biomimetic pattern that creates both physical interlocking with the orthopedic brace and enhanced surface tension forces.

2. The compression sleeve of claim 1, wherein the gripping component comprises an elastomer coated onto at least a portion of the outside surface of the tubular body.

3. The compression sleeve of claim 2, wherein the elastomer comprises a thermoplastic elastomer selected from the group consisting of a styrenic block copolymer, a thermoplastic olefin, an elastomeric alloy, a thermoplastic polyurethane, a thermoplastic copolyester, a thermoplastic polyamide, and combinations thereof.

4. The compression sleeve of claim 2, wherein the elastomer is selected from the group consisting of a natural polyisoprene, a synthetic polyisoprene, a polybutadiene, a chloroprene rubber, a butyl rubber, a halogenated butyl rubber, a styrene-butadiene rubber, a nitrile rubber, a hydrogenated nitrile rubber, an ethylene propylene rubber, an ethylene propylene diene rubber, an epichlorohydrin rubber, a polyacrylic rubber, a silicone rubber, a fluorosilicone rubber, a fluoroelastomer, a perfluoroelastomer, a polyether block amide, a chlorosulfonated ethylene-vinyl acetate, a resilin, an elastin, a polysulfide rubber, latex, an elastolefin, and combinations thereof.

5. The compression sleeve of claim 1, wherein the inside surface of the tubular body includes a gripping component at an upper portion thereof to prevent the tubular body from sliding down the human limb.

6. The compression sleeve of claim 1, wherein the tubular body has a thickness ranging from 0.2 mm to 5.0 mm.

7. The compression sleeve of claim 1, wherein the tubular body is fabricated from a material selected from the group consisting of cotton, wool, linen, silk, cashmere, hemp, jute, elastane, nylon, modacrylic, olefin, acrylic, polyester, rayon, vinyon, saran, lycra, spandex, vinalon, aramids, modal, dyneema/spectra, polybenzimidazole fiber, sulfar, lyocell, orlon, zylon, vectran, derclon, and combinations thereof.

8. The compression sleeve of claim 1, wherein the gripping component comprises a pattern of raised elastomeric elements distributed across the outside surface of the tubular body in an anisotropic lattice structure that provides greater resistance to axial forces than to circumferential expansion forces.

9. The compression sleeve of claim 1, wherein the micro-channeled surface structure comprises elastomeric ridges having a width between 50 and 200 micrometers and a depth between 25 and 100 micrometers.

10. The compression sleeve of claim 1, wherein the tubular body comprises embedded shape-memory polymer filaments having diameters between 0.1 mm and 0.5 mm distributed in a helical pattern throughout the sleeve, the filaments programmed to create dynamic, persistent compression that adapts to changes in swelling throughout a healing process.

11. A treatment assembly, comprising: a first layer comprising an area of a human body; a compression sleeve positioned over the first layer, the compression sleeve comprising a generally tubular body having an outside surface and an inside surface, wherein the outside surface includes at least one gripping component; and an orthopedic brace positioned over the compression sleeve, wherein the gripping component engages with the orthopedic brace to prevent the orthopedic brace from sliding along or rotating around the compression sleeve, wherein the gripping component comprises variable-density grip zones that correspond to specific structural elements of the orthopedic brace to create targeted resistance at interfaces most prone to slippage.

12. The treatment assembly of claim 11, further comprising: a second layer positioned between the first layer and the compression sleeve, wherein the second layer comprises a non-adherent gauze for absorbing bodily fluids, the gauze having antimicrobial properties and a thickness between 0.5 mm and 3 mm; and a third layer positioned between the second layer and the compression sleeve, wherein the third layer comprises an elastic bandage applied with 20-30% stretch at a distal portion decreasing to 10-15% stretch at a proximal end to create a therapeutic pressure gradient.

13. The treatment assembly of claim 12, wherein the elastic bandage comprises longitudinal tensile indicators that visually confirm proper tensioning during application.

14. The treatment assembly of claim 11, wherein the compression sleeve is configured to provide therapeutic compression to the area of the human body.

15. The treatment assembly of claim 11, wherein the orthopedic brace provides structural support to the area of the human body.

16. A method of stabilizing an orthopedic brace on a limb of a patient, comprising the steps of: applying a compression sleeve over the limb, the compression sleeve comprising a tubular body having an outside surface and an inside surface, wherein the outside surface includes at least one gripping component; positioning an orthopedic brace over the compression sleeve such that the gripping component engages with the orthopedic brace; and securing the orthopedic brace in position, wherein the engagement between the gripping component and the orthopedic brace prevents the orthopedic brace from sliding along or rotating around the limb during patient movement, wherein the gripping component comprises a micro-channeled surface structure comprising an array of microscopic elastomeric ridges that creates physical interlocking with the orthopedic brace.

17. The method of claim 16, wherein prior to applying the compression sleeve, the method further comprises: applying a non-adherent gauze layer to the limb; and applying an elastic bandage over the gauze layer with a pressure gradient, wherein the elastic bandage is applied with 20-30% stretch at a distal portion decreasing to 10-15% stretch at a proximal end.

18. The method of claim 16, further comprising: performing a movement test to verify that the orthopedic brace remains stable during a range of motion of the limb.

19. The method of claim 16, wherein the inside surface of the compression sleeve includes a gripping component at an upper portion thereof, and wherein applying the compression sleeve includes positioning the gripping component on the inside surface to prevent the compression sleeve from sliding down the limb.

20. The method of claim 16, wherein the Shore A hardness of the gripping component is calibrated to be 15-25 points lower than the hardness of the orthopedic brace material, creating an optimal hardness differential for mechanical interlocking between the compression sleeve and the orthopedic brace.
